(19) United States
(12) Patent Application Publication
(10) Pub. No.: US 2026/0051027 A1
(43) Pub. Date: Feb. 19, 2026

(54) GENERATION OF SYNTHETIC CONTRAST-ENHANCED RADIOLOGICAL IMAGES

(71) Applicant: BAYER AKTIENGESELLSCHAFT, Leverkusen (DE)

(72) Inventors: Johannes HÖHNE, Oranienburg (DE); Felix Karl KREIS, Berlin (DE); Parvaneh JANBAKHSHI, Berlin (DE); Ivo Matteo BALTRUSCHAT, Hamburg (DE)

(21) Appl. No.: 19/233,600

(22) Filed: Jun. 10, 2025

(30) Foreign Application Priority Data
Jun. 10, 2024 (EP) 24181079.5
Jun. 18, 2024 (EP) 24182725.2

(51) Int. Cl. G06T 5/50; A61B 6/00; A61K 49/00; G01R 33/28; G06T 5/94

(52) U.S. Cl. CPC G06T 5/50; A61B 6/481; A61K 49/0002; G01R 33/282; G06T 5/94; A61K 2123/00; G06T 2207/10081; G06T 2207/10088; G06T 2207/20224; G06T 2207/30056; G06T 2207/30061

(57) ABSTRACT

The systems, methods and computer programs disclosed herein relate to the generation of synthetic contrast-enhanced radiological images.

### CROSS-REFERENCE TO RELATED APPLICATION

This application claims the benefit of, and priority to, European Patent Application No. 24181079.5, filed on Jun. 10, 2024, and European Patent Application No. 24182725.2, filed on Jun. 18, 2024. The entire disclosures of the above-referenced applications are incorporated herein by reference.

### TECHNICAL FIELD

The systems, methods and computer programs disclosed herein relate to the generation of synthetic contrast-enhanced radiological images.

### INTRODUCTION (BACKGROUND)

WO2024/100233A1 discloses a method for generating a synthetic contrast-enhanced radiological image of an examination region of an examination object. The synthetic contrast-enhanced radiological image is generated on the basis of a first and a second radiological image of the examination region of the examination object. The first radiological image represents the examination region without contrast agent. The second radiological image represents the examination region after administration of a contrast agent. Subtraction of the first radiological image from the second radiological image generates a representation of the examination region representing the spread of contrast agent in the examination region. This representation is multiplied by an amplification factor and the result of the multiplication is added to the first radiological image. The result is a synthetic contrast-enhanced radiological image in which the signal attributable to contrast agent in the examination region is amplified relative to the second radiological image.

One problem with the method disclosed in WO2024/100233A1 is that noise is amplified to the same degree as the signal attributable to the contrast agent.

This problem is addressed by the subjects of the present disclosure.

### SUMMARY

The present disclosure provides, in a first aspect, a computer-implemented method comprising the steps of:
providing a first representation, where the first representation represents an examination region of an examination object without contrast agent or after administration of a first amount of a contrast agent,
providing a second representation, where the second representation represents the examination region of the examination object after administration of a second amount of the contrast agent,
generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
generating a fourth representation of the examination region of the examination object, where the generation of the fourth representation comprises non-linear transformation of at least a portion of the difference values of the third representation,
generating a synthetic contrast-enhanced representation of the examination region of the examination object, where the generation of the synthetic contrast-enhanced representation comprises addition of the fourth representation to the first representation or to the second representation,
outputting and/or storing the synthetic contrast-enhanced representation of the examination region of the examination object and/or transmitting the synthetic contrast-enhanced representation to a separate computer system.

The present disclosure further provides a computer system comprising:
a processor; and
a memory that stores a computer program configured to perform an operation when executed by the processor, said operation comprising the steps of:
   providing a first representation, where the first representation represents an examination region of an examination object without contrast agent or after administration of a first amount of a contrast agent,
   providing a second representation, where the second representation represents the examination region of the examination object after administration of a second amount of the contrast agent,
   generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
   generating a fourth representation of the examination region of the examination object, where the generation of the fourth representation comprises non-linear transformation of at least a portion of the difference values of the third representation,
   generating a synthetic contrast-enhanced representation of the examination region of the examination object, where the generation of the synthetic contrast-enhanced representation comprises addition of the fourth representation to the first representation or to the second representation,
   outputting and/or storing the synthetic contrast-enhanced representation of the examination region of the examination object and/or transmitting the synthetic contrast-enhanced representation to a separate computer system.

The present disclosure further provides a computer program that can be loaded into a working memory of a computer system, where it causes the computer system to execute the following steps:
providing a first representation, where the first representation represents an examination region of an examination object without contrast agent or after administration of a first amount of a contrast agent,
providing a second representation, where the second representation represents the examination region of the examination object after administration of a second amount of the contrast agent,
generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
generating a fourth representation of the examination region of the examination object, where the generation of the fourth representation comprises non-linear transformation of at least a portion of the difference values of the third representation,
generating a synthetic contrast-enhanced representation of the examination region of the examination object, where the generation of the synthetic contrast-enhanced representation comprises addition of the fourth representation to the first representation or to the second representation,
outputting and/or storing the synthetic contrast-enhanced representation of the examination region of the examination object and/or transmitting the synthetic contrast-enhanced representation to a separate computer system.

The present disclosure further provides for a use of a contrast agent in a radiological examination method, wherein the radiological examination method comprises the steps of:
generating a first representation, where the first representation represents an examination region of an examination object without contrast agent or after administration of a first amount of the contrast agent,
generating a second representation, where the second representation represents the examination region of the examination object after administration of a second amount of the contrast agent,
generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
generating a fourth representation of the examination region of the examination object, where the generation of the fourth representation comprises non-linear transformation of at least a portion of the difference values of the third representation,
generating a synthetic contrast-enhanced representation of the examination region of the examination object, where the generation of the synthetic contrast-enhanced representation comprises addition of the fourth representation to the first representation or to the second representation,
outputting and/or storing the synthetic contrast-enhanced representation of the examination region of the examination object and/or transmitting the synthetic contrast-enhanced representation to a separate computer system.

The present disclosure further provides a contrast agent for use in a radiological examination method, wherein the radiological examination method comprises the steps of:
generating a first representation, where the first representation represents an examination region of an examination object without contrast agent or after administration of a first amount of the contrast agent,
generating a second representation, where the second representation represents the examination region of the examination object after administration of a second amount of the contrast agent,
generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
generating a fourth representation of the examination region of the examination object, where the generation of the fourth representation comprises non-linear transformation of at least a portion of the difference values of the third representation,
generating a synthetic contrast-enhanced representation of the examination region of the examination object, where the generation of the synthetic contrast-enhanced representation comprises addition of the fourth representation to the first representation or to the second representation,
outputting and/or storing the synthetic contrast-enhanced representation of the examination region of the examination object and/or transmitting the synthetic contrast-enhanced representation to a separate computer system.

The present disclosure further provides a kit comprising a computer program product and a contrast agent, wherein the computer program product comprises a computer program that can be loaded into a working memory of a computer system, where it causes the computer system to execute the following steps:
providing a first representation, where the first representation represents an examination region of an examination object without contrast agent or after administration of a first amount of the contrast agent,
providing a second representation, where the second representation represents the examination region of the examination object after administration of a second amount of the contrast agent,
generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
generating a fourth representation of the examination region of the examination object, where the generation of the fourth representation comprises non-linear transformation of at least a portion of the difference values of the third representation,
generating a synthetic contrast-enhanced representation of the examination region of the examination object, where the generation of the synthetic contrast-enhanced representation comprises addition of the fourth representation to the first representation or to the second representation,
outputting and/or storing the synthetic contrast-enhanced representation of the examination region of the examination object and/or transmitting the synthetic contrast-enhanced representation to a separate computer system.

Further subjects and embodiments can be found in the dependent claims, the present description and the drawings.

### BRIEF DESCRIPTION OF THE DRAWINGS

FIG. 1 shows by way of example and in schematic form a non-linear transformation according to the present disclosure.

FIG. 2 shows in schematic form a further example of a non-linear transformation according to the present disclosure.

FIG. 3 shows by way of example and in schematic form the generation of a synthetic contrast-enhanced representation.

FIG. 4 shows by way of example and in schematic form a further embodiment of the generation of a synthetic contrast-enhanced representation.

FIG. 5 shows by way of example and in schematic form a method for generating a synthetic contrast-enhanced representation in the form of a flow chart.

FIG. 6 shows by way of example and in schematic form a computer system according to the present disclosure.

FIG. 7 shows by way of example and in schematic form a further embodiment of the computer system of the present disclosure.

FIG. 8 shows a comparison of the contrast-to-noise ratios of synthetic contrast-enhanced representations that were generated by different methods.

### DETAILED DESCRIPTION

The subjects of the present disclosure will be more particularly elucidated below, without distinguishing between the subjects (method, computer system, computer program (product), use, contrast agent for use, kit). Rather, the elucidations that follow are intended to apply analogously to all subjects, irrespective of the context (method, computer system, computer program (product), use, contrast agent for use, kit) in which they occur.

Where steps are stated in an order in the present description or in the claims, this does not mean that this disclosure is limited to the order stated. Instead, it is conceivable that the steps are also executed in a different order or else in parallel with one another, the exception being when one step builds, for example, on another step, thereby making it necessary that the step building on the previous step be executed next (which will however become clear in the individual case). The orders stated thus constitute illustrative embodiments.

In certain places, the subjects of the present disclosure will be more particularly elucidated with reference to drawings. The drawings show specific embodiments having specific features and combinations of features that are primarily for purposes of illustration; the disclosure is not to be understood as being limited to the features and combinations of features shown in the drawings. Furthermore, statements made in the description of the drawings in relation to features and combinations of features are intended to be generally applicable, that is to say applicable to other embodiments too and not limited to the embodiments shown.

The embodiments described and/or mentioned in this disclosure may be combined with each other, unless they are mutually exclusive. In other words, the present disclosure also includes any combination of non-exclusive embodiments.

The present disclosure describes means for generating a synthetic radiological image of an examination region of an examination object.

The term “synthetic” means that the synthetic radiological image is not the (direct) result of a measurement on an actual examination object, but that the synthetic radiological image is the result of calculations. A synonym for the term “synthetic” is the term “artificial”. However, a synthetic radiological image may be based on measured radiological images, i.e. the calculations may be made on the basis of measured radiological images.

The “examination object” is normally a living being, for example a mammal, for example a human.

The “examination region” is a part of the examination object, for example an organ or part of an organ or a plurality of organs or another part of the examination object.

For example, the examination region may be a liver, kidney, heart, lung, brain, stomach, bladder, pancreas, prostate, breast, intestine or a part thereof or another part of the body of a mammal (for example a human).

In one embodiment, the examination region includes a liver or part of a liver or the examination region is a liver or part of a liver of a mammal, for example a human.

In a further embodiment, the examination region includes a brain or part of a brain or the examination region is a brain or part of a brain of a mammal, for example a human.

In a further embodiment, the examination region includes a heart or part of a heart or the examination region is a heart or part of a heart of a mammal, for example a human.

In a further embodiment, the examination region includes a thorax or part of a thorax or the examination region is a thorax or part of a thorax of a mammal, for example a human.

In a further embodiment, the examination region includes a stomach or part of a stomach or the examination region is a stomach or part of a stomach of a mammal, for example a human.

In a further embodiment, the examination region includes a pancreas or part of a pancreas or the examination region is a pancreas or part of a pancreas of a mammal, for example a human.

In a further embodiment, the examination region includes a kidney or part of a kidney or the examination region is a kidney or part of a kidney of a mammal, for example a human.

In a further embodiment, the examination region includes one or both lungs or part of a lung of a mammal, for example a human.

In a further embodiment, the examination region includes a breast or part of a breast or the examination region is a breast or part of a breast of a female mammal, for example a female human.

In a further embodiment, the examination region includes a prostate or part of a prostate or the examination region is a prostate or part of a prostate of a male mammal, for example a male human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

In a first step, a first representation and a second representation of the examination region of the examination object are provided.

The term “providing” can for example denote “receiving” or “generating”.

The term “receiving” encompasses both the retrieving and the accepting of representations transmitted for example to the computer system of the present disclosure. Representations may be read from one or more data memories and/or transmitted from a separate computer system. Representations may be received for example from a computed tomography system, from a magnetic resonance imaging system, from a positron emission tomography system or from an ultrasound scanner.

The term “generating” can mean that a representation is generated on the basis of another (for example a received) representation or on the basis of a plurality of other (for example received) representations. For example, a received representation may be a representation of an examination region of an examination object in frequency space. On the basis of this frequency-space representation, it is possible for example to generate a representation of the examination region of the examination object in real space through a transformation (for example an inverse Fourier transform). Other ways of generating a representation on the basis of one or more other representations are described in this description.

The term “generating” can also mean that a representation is generated by measurement. A generated representation may thus be the result of a radiological examination on an examination object.

In one embodiment of the present disclosure, the first representation and/or the second representation are the result of a radiological examination on an examination object.

“Radiology” is the branch of medicine that is concerned with the use of electromagnetic rays and mechanical waves (including for instance ultrasound diagnostics) for diagnostic, therapeutic and/or scientific purposes. Besides X-rays, other ionizing radiations such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present disclosure encompasses in particular the following examination methods: computed tomography (CT), magnetic resonance imaging (MRI), positron emission tomography (PET), sonography.

In one embodiment of the present disclosure, the radiological examination is a magnetic resonance imaging examination. In one embodiment of the present disclosure, the first representation and the second representation are the result of a magnetic resonance imaging examination. The first representation and the second representation may be MRI images or may have been generated from one or more MRI images.

In a further embodiment, the radiological examination is a computed tomography examination. In one embodiment of the present disclosure, the first representation and the second representation are the result of a computed tomography examination. The first representation and the second representation may be CT images or may have been generated from one or more CT images.

In a further embodiment, the radiological examination is a PET examination. In one embodiment of the present disclosure, the first representation and the second representation are the result of a PET examination. The first representation and the second representation may be PET images or may have been generated from one or more PET images.

In a further embodiment, the radiological examination is an ultrasound examination. In one embodiment of the present disclosure, the first representation and the second representation are the result of an ultrasound examination. The first representation and the second representation may be ultrasound images or may have been generated from one or more ultrasound images.

The first representation and the second representation may be a representation in real space (image space), a representation in frequency space, a representation in projection space or a representation in some other space.

In a representation in real space, also referred to in this description as real-space depiction or real-space representation, the examination region is normally represented by a large number of image elements (for example pixels or voxels or doxels) that may for example be in a raster arrangement, in which case each image element represents a part of the examination region and each image element may be assigned a colour value or grey value. The colour value or grey value normally represents a signal intensity, for example the attenuation of X-rays. A format widely used in radiology for storing and processing representations in real space is the DICOM format. DICOM (Digital Imaging and Communications in Medicine) is an open standard for storing and exchanging information in medical image data management.

In a representation in frequency space, also referred to in this description as frequency-space depiction or frequency-space representation, the examination region is represented by a superposition of fundamental frequencies. For example, the examination region may be represented by a sum of sine and cosine functions having different amplitudes, frequencies and phases. The amplitudes and phases may be plotted as a function of the frequencies, for example, in a two-or three-dimensional representation. Normally, the lowest frequency (origin) is placed in the centre. The further away from this centre, the higher the frequencies. Each frequency can be assigned an amplitude representing the frequency in the frequency-space depiction and a phase indicating the extent of the shift of the respective wave with respect to a sine or cosine wave.

A representation in real space can for example be converted (transformed) by a Fourier transform into a representation in frequency space. Conversely, a representation in frequency space can for example be converted (transformed) by an inverse Fourier transform into a representation in real space.

Details about real-space depictions and frequency-space depictions and their respective interconversion are described in numerous publications, see for example https://see.stanford.edu/materials/lsoftace261/book-fall-07.pdf.

A representation of an examination region in projection space is normally the result of a computed tomography examination prior to image reconstruction. In other words: the raw data obtained in the computed tomography examination can be understood as a projection-space depiction. In computed tomography, the intensity or attenuation of X-radiation as it passes through the examination object is measured. From this, projection values can be calculated. In a second step, the object information encoded by the projection is transformed into an image (real-space depiction) through a computer-aided reconstruction. The reconstruction can be effected with the Radon transform. The Radon transform describes the link between the unknown examination object and its associated projections.

Details about the transformation of projection data into a real-space depiction are described in numerous publications, see for example K. Fang: The Radon Transformation and Its Application in Tomography, Journal of Physics Conference Series 1903(1):012066.

There are further spaces in which it is possible for there to be representations of the examination region. For the sake of simplicity and better clarity, the subjects of the present disclosure are in large parts of the description described on the basis of real-space representations. This should not however be understood as limiting. Those skilled in the art of image analysis know how to apply the appropriate parts of the description to representations other than real-space representations.

The first representation represents the examination region of the examination object without contrast agent or after administration of a first amount of a contrast agent. The second representation represents the examination region of the examination object after administration of a second amount of the contrast agent.

“Contrast agents” are substances or mixtures of substances that improve the depiction of structures and functions of the body in radiological examinations.

In computed tomography, iodine-containing solutions are normally used as contrast agents. In magnetic resonance imaging (MRI), superparamagnetic substances (e.g. iron oxide nanoparticles, superparamagnetic iron-platinum particles (SIPPs)) or paramagnetic substances (e.g. gadolinium chelates, manganese chelates) are usually used as contrast agents. In the case of sonography, liquids containing gas-filled microbubbles are normally administered intravenously. In the case of positron emission tomography (PET), radiotracers are used as contrast agents.

Examples of contrast agents can be found in the literature (see for example A. S. L. Jascinth et al.: Contrast Agents in computed tomography: A Review, Journal of Applied Dental and Medical Sciences, 2016. Vol. 2. Issue 2. 143-149; H. Lusic et al.: X-ray-Computed Tomography Contrast Agents, Chem. Rev. 2013, 113, 3, 1641-1666; https://www.radiology.wise.edu/wp-content/uploads/2017/10/contrast-agents-tutorial.pdf, M. R. Nough et al.: Radiographic and magnetic resonances contrast agents: Essentials and tips for safe practices, World J Radiol. 2017 Sep. 28; 9(9): 339-349; L. C. Abonyi et al.: Intravascular Contrast Media in Radiography: Historical Development & Review of Risk Factors for Adverse Reactions, South American Journal of Clinical Research, 2016, Vol. 3. Issue 1, 1-10; ACR Manual on Contrast Media, 2020, ISBN: 978-1-55903-012-0; A. Ignee et al.: Ultrasound contrast agents, Endosc Ultrasound. 2016 November-December; 5(6): 355-362; J. Trotter et al.: Positron Emission Tomography (PET)/Computed Tomography (CT) Imaging in Radiation Therapy Treatment Planning: A Review of PET Imaging Tracers and Methods to Incorporate PET/CT, Advances in Radiation Oncology (2023) 8, 101212).

In one embodiment of the present invention, the contrast agent is an MRI contrast agent.

In one embodiment of the present disclosure, the contrast agent is an agent that includes gadolinium (III) 2-[4,7,10-tris (carboxymethyl)-1,4,7,10-tetrazacyclododec-1-yl] acetic acid (also referred to as gadolinium-DOTA or gadoteric acid).

In a further embodiment, the contrast agent is an agent that includes gadolinium (III) ethoxybenzyldiethylenetriaminepentaacetic acid (Gd-EOB-DTPA); preferably, the contrast agent includes the disodium salt of gadolinium (III) ethoxybenzyldiethylenetriaminepentaacetic acid (also referred to as gadoxetic acid).

In one embodiment of the present disclosure, the contrast agent is an agent that includes gadolinium (III) 2-[3,9-bis [1-carboxylato-4-(2,3-dihydroxypropylamino)-4-oxobutyl]-3,6,9,15-tetrazabicyclo [9.3.1] pentadeca-1(15),11, 13-trien-6-yl]-5-(2,3-dihydroxypropylamino)-5-oxopentanoate (also referred to as gadopiclenol) (see for example WO2007/042504 and WO2020/030618 and/or WO2022/013454).

In one embodiment of the present disclosure, the contrast agent is an agent that includes dihydrogen [(±)-4-carboxy-5,8,11-tris (carboxymethyl)-1-phenyl-2-oxa-5,8,11-triazatridecan-13-oato (5-)]gadolinate (2-) (also referred to as gadobenic acid).

In one embodiment of the present disclosure, the contrast agent is an agent that includes tetragadolinium [4,10-bis (carboxylatomethyl)-7-{3,6,12,15-tetraoxo-16-[4,7,10-tris-(carboxylatomethyl)-1,4,7,10-tetraazacyclododecan-1-yl]-9,9-bis ({ [({2-[4,7,10-tris-(carboxylatomethyl)-1,4,7,10-tetraazacyclododecan-1-yl]propanoyl}amino) acetyl]amino}methyl)-4,7, 11, 14-tetraazaheptadecan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57 (10): 629-638; WO2016193190).

In one embodiment of the present disclosure, the contrast agent is an agent that includes a Gd³⁺ complex of a compound of the formula (I)

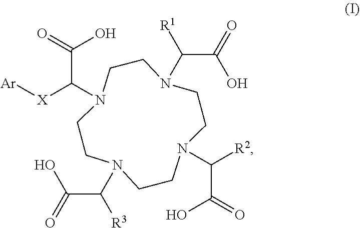

where
Ar is a group selected from

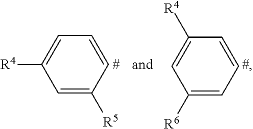

where # is the linkage to X,
X is a group selected from
CH₂, (CH₂)₂, (CH₂)₃, (CH₂)₄ and *—(CH₂)₂—O—CH₂-^{#},
where * is the linkage to Ar and # is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof.

In one embodiment of the present disclosure, the contrast agent is an agent that includes a Gd³⁺ complex of a compound of the formula (II)

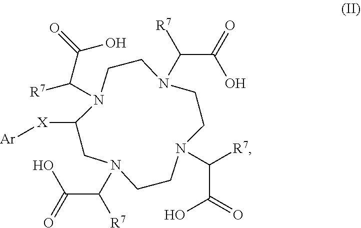

where
Ar is a group selected from

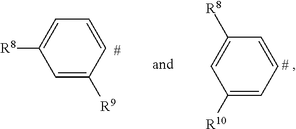

where # is the linkage to X,
X is a group selected from CH₂, (CH₂)₂, (CH₂)₃, (CH₂)₄ and * —(CH₂)₂—O—CH₂—^{#}, where * is the linkage to Ar and # is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

The term “C₁-C₃ alkyl” denotes a linear or branched. saturated monovalent hydrocarbon group having 1, 2 or 3 carbon atoms, for example methyl, ethyl, n-propyl or isopropyl. The term “C₂-C₄ alkyl” denotes a linear or branched, saturated monovalent hydrocarbon group having 2, 3 or 4 carbon atoms.

The term “C₂-C₄ al koxy” denotes a linear or branched, saturated monovalent group of the formula (C₂-C₄ alkyl)-O-, in which the term “C₂-C₄ alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-cthoxyphenyl)ethoxy]ethyl}-1,4,7,10-tetraazacyclododecane-1,4,7-triyl) triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris (3-hydroxypropanoate) (see for example WO2022/194777. example 15).

In one embodiment of the present disclosure, the contrast agent is an agent that includes gadolinium 2,2′,2″-{10- [(1S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium 2,2′,2″-{(2S)-10-(carboxymethyl)-2-[4-(2-ethoxyethoxy) benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium (III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxocthyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

In one embodiment of the present invention, the contrast agent is a hepatobiliary contrast agent. A hepatobiliary contrast agent has the characteristic features of being specifically taken up by liver cells (hepatocytes), accumulating in the functional tissue (parenchyma) and enhancing contrast in healthy liver tissue. An example of a hepatobiliary contrast agent is the disodium salt of gadoxetic acid (Gd-EOB-DTPA disodium), which is described in U.S. Pat. No. 6,039,931A and is commercially available under the trade names Primovist® and Eovist®. Further hepatobiliary contrast agents are described inter alia in WO2022/194777.

In one embodiment, the radiological examination is an MRI examination in which an MRI contrast agent is used.

In a further embodiment, the radiological examination is a CT examination in which a CT contrast agent is used.

In a further embodiment, the radiological examination is a CT examination in which an MRI contrast agent is used.

The first representation represents the examination region without contrast agent or after administration of a first amount of a contrast agent. In one embodiment, the first representation represents the examination region without contrast agent.

The second representation represents the examination region after administration of a second amount of a contrast agent. The second amount is larger than the first amount (which, as described, may also be zero). The expression “after administration of a second amount of the contrast agent” should not be understood as meaning that the first amount and the second amount add up in the examination region (unless the first amount is zero). Thus, the expression “the representation represents the examination region after administration of a (first or second) amount” should rather be understood as meaning: “the representation represents the examination region with a (first or second) amount” or “the representation represents the examination region including a (first or second) amount”.

In one embodiment, both the first amount and the second amount of the contrast agent are smaller than the standard amount.

In a further embodiment, the second amount of the contrast agent corresponds to the standard amount.

In a further embodiment, the first amount of the contrast agent is equal to zero and the second amount of the contrast agent is smaller than the standard amount.

In a further embodiment, the first amount of the contrast agent is equal to zero and the second amount of the contrast agent corresponds to the standard amount.

The standard amount is normally the amount recommended by the manufacturer and/or distributor of the contrast agent and/or the amount authorized by a regulatory authority and/or the amount specified in a package leaflet for the contrast agent.

For example, the standard amount of Primovist® is 0.025 mmol Gd-EOB-DTPA disodium/kg body weight.

On the basis of the first representation and the second representation, a third representation is generated.

The generation of the third representation comprises subtraction of the first representation from the second representation. The subtraction of the first representation from the second representation may for example be done in real space. In such a case, both the first representation and the second representation are in the form of real-space representations. The first representation and the second representation each comprise a multiplicity of image elements. The term “multiplicity of image elements” means at least 100 and usually more than 1000 image elements. Each image element represents a subregion of the examination region of the examination object. Each image element is normally assigned at least one colour value or one grey value. The subtraction of the first representation from the second representation is normally done image element by image element. In this case, the colour values or grey values of corresponding image elements are subtracted from one another. “Corresponding image elements” are those that represent the same subregion of the examination region.

The third representation likewise comprises a multiplicity of image elements. Each image element is assigned a difference value. The difference value is the result of subtraction of the colour values or grey values of corresponding image elements of the first representation and second representation from each other.

It is possible to set negative difference values to zero (or some other fixed value) in order to avoid negative difference values.

If the subtraction of the first representation from the second representation is done in frequency space. the generation of the third representation further comprises: transformation of the result of the subtraction into real space. Such a transformation may for example be done by means of an inverse Fourier transform.

On the basis of the third representation, a fourth representation is generated. The generation of the fourth representation comprises non-linear transformation of at least a portion of the difference values of the third representation.

The non-linear transformation may be an amplification or attenuation of difference values.

The term “amplification” means that the magnitude of a difference value (i.e. the absolute value of the difference value) is increased. The term “attenuation” means that the magnitude of a difference value (i.e. the absolute value of the difference value) is decreased.

In the method disclosed in WO2024/100233A1, each difference value is multiplied by a constant amplification factor. Here. “constant” means that the amplification factor is always the same irrespective of the respective difference value. In the case of the present disclosure, the transformation (amplification/attenuation), at least for a portion of the difference values, depends on the size of the respective difference value.

The embodiments listed in this disclosure in connection with the transformation and/or amplification and/or attenuation of difference values may be combined with each other as desired, provided they are not mutually exclusive. This applies in particular to the following embodiments. Therefore, the present disclosure thus also includes combinations of the (following) embodiments.

In one embodiment of the present disclosure, the amplification and/or attenuation (increase and/or decrease in magnitudes) of difference values depends only on the size of the difference values (and not, for example, on the position and/or configuration of the image elements).

In one embodiment of the present disclosure, all positive difference values are amplified, at least a portion of the positive difference values being non-linearly amplified.

In one embodiment of the present disclosure, negative difference values and positive difference values are amplified, at least a portion of the positive difference values being non-linearly amplified.

In one embodiment of the present disclosure, all positive difference values and all negative difference values are amplified, at least a portion of the positive difference values being non-linearly amplified.

In one embodiment of the present disclosure, negative difference values and positive difference values are amplified, at least a portion of the positive difference values and at least a portion of the negative difference values being non-linearly amplified.

In one embodiment of the present disclosure, all positive difference values and all negative difference values are amplified, at least a portion of the positive difference values being non-linearly amplified and at least a portion of the negative difference values being non-linearly amplified.

In one embodiment of the present disclosure, all positive difference values and all negative difference values are amplified, at least a portion of the positive difference values being non-linearly amplified and all negative difference values being non-linearly amplified.

In one embodiment of the present disclosure, each difference value may be assigned an amplification factor. This means that the amplification depends only on the respective difference value and not on the image element having the difference value. The amplification factor may also be an attenuation factor. i.e. in the case of difference values, it may lead to a decrease in the magnitude of the difference values.

In one embodiment of the present disclosure, there is an upper positive difference value, and difference values above the upper positive difference value are linearly amplified. The term “upper” refers to the size of the difference value. Difference values above the upper positive difference value are those that are greater than the upper positive difference value.

In one embodiment of the present disclosure, difference values that are zero remain unchanged. i.e. they are not amplified or attenuated, but remain zero.

In one embodiment of the present disclosure, there is an upper positive difference value, and difference values between the difference value zero and the upper positive difference value are non-linearly amplified. Difference values between the difference value zero and the upper positive difference value are those that are greater than zero and less than the upper positive difference value.

In one embodiment of the present disclosure, the amplification above an upper positive difference value is linear and the amplification in a range between the difference value zero and the upper positive difference value is non-linear and lower (less) than the linear amplification. This means that the non-linearly amplified difference value is less than if it were linearly amplified.

In one embodiment of the present disclosure, the amplification above an upper positive difference value is linear and the amplification in a range between the difference value zero and the upper positive difference value is non-linear and greater than the linear amplification. This means that the non-linearly amplified difference value is greater than if it were linearly amplified. In one embodiment of the present disclosure, there is a lower negative difference value, and difference values between the difference value zero and the lower negative difference value are non-linearly amplified. The term “lower negative difference value” refers to the size of the difference value. Difference values below the lower negative difference value are those with a magnitude (absolute value) greater than the magnitude of the lower negative difference value.

In one embodiment of the present disclosure, the amplification below a lower negative difference value is linear and the amplification in a range between the lower negative difference value and the difference value zero is non-linear and lower than the linear amplification. This means that the magnitude of the non-linearly amplified difference value is less than the magnitude of the correspondingly linearly amplified difference value.

In one embodiment of the present disclosure, the amplification below a lower negative difference value is linear and the amplification in a range between the lower negative difference value and the difference value zero is non-linear and greater than the linear amplification. This means that the magnitude of the non-linearly amplified difference value is greater than the magnitude of the correspondingly linearly amplified difference value.

In one embodiment of the present disclosure, all negative difference values are non-linearly amplified.

In one embodiment of the present disclosure, all difference values are amplified by a factor that depends on the respective difference value and is greater than or equal to one.

In one embodiment of the present disclosure, all positive difference values are amplified by a factor, the factor being zero for the difference value zero and assuming a maximum value for an upper positive difference value and assuming a minimum value for a difference value between the difference value zero and the upper positive difference value. The factors of the remaining positive difference values may assume values that correspond to the minimum value or the maximum value or are between the minimum value and the maximum value.

In one embodiment of the present disclosure, all negative difference values are amplified by a factor, the factor being zero for the difference value zero and assuming a maximum value for a lower negative difference value and assuming a minimum value for a difference value between the difference value zero and the lower negative difference value. The factors of the remaining negative difference values may assume values that correspond to the minimum value or the maximum value or are between the minimum value and the maximum value.

In one embodiment of the present disclosure, all difference values are amplified by a factor, the factor being zero for the difference value zero and assuming a maximum value for an upper absolute difference value and assuming a minimum value for an absolute difference value between the difference value zero and the upper absolute difference value. The factors of the remaining difference values may assume values that correspond to the minimum value or the maximum value or are between the minimum value and the maximum value.

In one embodiment of the present disclosure, all difference values are non-linearly amplified.

In one embodiment of the present disclosure, a defined portion of difference values is non-linearly amplified, whereas the remaining difference values are linearly amplified.

In one embodiment of the present disclosure, the difference values of the upper p-quantile of the positive difference values are linearly amplified, whereas the difference values of the lower p-quantile of the positive difference values are non-linearly amplified. Here, p may assume a value in the range from 0.5 to 0.99. In one embodiment of the present disclosure, p is in the range from 0.6 to 0.99. In one embodiment of the present disclosure, p is in the range from 0.7 to 0.99. In one embodiment of the present disclosure, p is in the range from 0.8 to 0.99. In one embodiment of the present disclosure, p is in the range from 0.9 to 0.99. In one embodiment of the present disclosure, p is in the range from 0.95 to 0.99.

In one embodiment of the present disclosure, the difference values of the upper p-quantile of the absolute difference values are linearly amplified, whereas the difference values of the lower p-quantile of the absolute difference values are non-linearly amplified. Here, p may assume a value in the range from 0.5 to 0.99. In one embodiment of the present disclosure, p is in the range from 0.6 to 0.99. In one embodiment of the present disclosure, p is in the range from 0.7 to 0.99. In one embodiment of the present disclosure, p is in the range from 0.8 to 0.99. In one embodiment of the present disclosure, p is in the range from 0.9 to 0.99. In one embodiment of the present disclosure, p is in the range from 0.95 to 0.99.

To form a p-quantile, all difference values may be sorted according to size. In the case of negative difference values, the absolute value (magnitude) can be formed before sorting. In the case of a p-quantile with p=0.5. the difference values are divided into two groups. Each group comprises 50% of the difference values. All difference values within the lower 0.5 quantile are less than the smallest difference value of the upper 0.5 quantile. All difference values within the upper 0.5 quantile are greater than the greatest difference value of the lower 0.5 quantile. In the case of a p-quantile with p=0.99, the difference values are likewise divided into two groups. The lower 0.99 quantile comprises 99% of the difference values. The upper 0.99 quantile comprises 1% of the difference values. All difference values within the lower 0.99 quantile are less than the smallest difference value of the upper 0.99 quantile. All difference values within the upper 0.99 quantile are greater than the greatest difference value of the lower 0.99 quantile.

In one embodiment of the present disclosure, the difference values are transformed by means of a transformation function, the transformation function comprising, at least within a range of difference values, a power function of the respective difference value.

In one embodiment of the present disclosure, the difference values are transformed by means of a transformation function, the transformation function comprising a continuous and continuously differentiable function at least within a range of difference values.

In one embodiment of the present disclosure, the difference values are transformed by means of a transformation function, the transformation function being a continuous and continuously differentiable function within a range extending from a lower negative difference value to an upper positive difference value.

In one embodiment of the present disclosure, at least a portion of the difference values are transformed by means of a transformation function. The transformation function comprises a transformation function ƒ⁽⁺⁾ that transforms positive difference values and a transformation function ƒ⁽⁻⁾ that transforms negative difference values, where:

$${f}^{( - )} ( {d}^{( - )} ) = - {f}^{( + )} ( \vert {d}^{( - )} \vert ) \qquad ( 1 )$$

Here, d⁽⁻⁾ is a negative difference value that is transformed and |d⁽⁻⁾51 is the magnitude of the negative difference value. In other words, determination of the amplified/attenuated value of a negative difference value (d⁽⁻⁾<0) using the transformation function ƒ⁽⁻⁾ can be achieved by also forming the magnitude |d⁽⁻⁾| of the negative difference value, applying the transformation function ƒ⁽⁺⁾ to said magnitude and multiplying the result by a factor of −1.

In one embodiment of the present disclosure, the difference values within a range of difference values are amplified by a transformation function, the transformation function having the following form:

$${f}^{( + )} = {β}^{( + )} \cdot {d}^{( + )} \cdot {( \frac{{d}^{( + )}}{{d}_{max}^{( + )}} )}^{{γ}^{( + )}} + {δ}^{( + )} \qquad ( 2 a )$$

$${f}^{( - )} = {β}^{( - )} \cdot {d}^{( - )} \cdot {( \frac{\vert {d}^{( - )} \vert}{{\vert d ( - ) \vert}_{max}} )}^{{γ}^{( - )}} + {δ}^{( - )} \qquad ( 2 b )$$

ƒ⁽⁺⁾ is a transformation function that transforms (amplifies/attenuates) positive difference values. ƒ⁽⁻⁾ is a transformation function that transforms (amplifies/attenuates) negative difference values.

β⁽⁺⁾ and β⁽⁻⁾ are factors that are normally greater than 1. β⁽⁺⁾ and β⁽⁻⁾ may assume, for example, a value between 1 and 10. They may also be greater than 10. In one embodiment, β⁽⁺⁾ and β⁽⁻⁾ are factors in the range from 1 to 5. In one embodiment, β⁽⁺⁾ and β⁽⁻⁾ are factors in the range from 1 to 4. In one embodiment, β⁽⁺⁾ and β⁽⁻⁾ are factors in the range from 1 to 3. In one embodiment, β⁽⁺⁾ and β⁽⁻⁾ are factors in the range from 1 to 2.5. In one embodiment, β⁽⁺⁾ and β⁽⁻⁾ are factors in the range from 1 to 2. β⁽⁺⁾ and β⁽⁻⁾ may be identical or different. In one embodiment, β⁽⁺⁾ and β⁽⁻⁾ are identical.

d⁽⁺⁾ is a positive difference value that is transformed.

$${d}_{max}^{( + )}$$

is the maximum positive difference value that occurs in the range of difference values.

The quotient

$$( \frac{{d}^{( + )}}{{d}_{max}^{( + )}} )$$

assumes a value of 1 if the positive difference value d⁽⁺⁾ that is transformed is equal to the maximum positive difference value

$${d}_{max}^{( + )} ,$$

otherwise the quotient is less than 1 and greater than 0.

d⁽⁻⁾ is a negative difference value that is transformed. |d⁽⁻⁾| is the magnitude (absolute value) of the negative difference value. |d⁽⁻⁾|ₘₐₓ is the maximum magnitude that occurs in the range of negative difference values.

The quotient

$$( \frac{\vert {d}^{( - )} \vert}{{\vert d ( - ) \vert}_{max}} )$$

assumes a value of 1 if the magnitude of the negative difference value |d⁽⁻⁾| that is transformed is equal to the maximum magnitude |d⁽⁻⁾|ₘₐₓ, otherwise the quotient is less than 1 and greater than 0.

The values

$${d}_{max}^{( + )}$$

and |d⁽⁻⁾|ₘₐₓ may be absolute values predefined by a user, for example. The values

$${d}_{max}^{( + )}$$

and |d⁽⁻⁾|ₘₐₓ may be values that are calculated from the available difference values (e.g. by determination of p-quantiles as described above).

γ⁽⁺⁾ and γ⁽⁻⁾ are exponents that may normally assume positive real values. If γ⁽⁺⁾ and γ⁽⁻⁾ are equal to 1. there is a linear amplification. If γ⁽⁺⁾ and γ⁽⁻⁾ are greater than zero and less than 1, then the amplification in the range of difference values under consideration is greater than the linear amplification. If γ⁽⁺⁾ and γ⁽⁻⁾ are greater than 1. then the amplification in the range of difference values under consideration is less than the linear amplification or there is an attenuation. γ⁽⁺⁾ and γ⁽⁻⁾ may be identical or different. In one embodiment, γ⁽⁺⁾ and γ⁽⁻⁾ are identical. In one embodiment, γ⁽⁺⁾ and γ⁽⁻⁾ are greater than 1. γ⁽⁺⁾ and γ⁽⁻⁾ may for example assume a value in the range from 1.1 to 3, or a value in the range from 1.2 to 2.9, or a value in the range from 1.3 to 2.8, or a value in the range from 1.4 to 2.7. or a value in the range from 1.5 to 2.6.

δ⁽⁺⁾ and δ⁽⁻⁾ are positive or negative real values. δ⁽⁺⁾ and δ⁽⁻⁾ may also be zero. δ⁽⁺⁾ and δ⁽⁻⁾ may be identical or different. In one embodiment. δ⁽⁺⁾ and δ⁽⁻⁾ are identical.

FIG. 1 shows by way of example and in schematic form a non-linear transformation according to the present disclosure.

Difference values d are transformed by means of a transformation function ƒ(d). In FIG. 1, the transformed difference values are plotted as a function ƒ(d) of the difference values d in a graphical representation in a Cartesian coordinate system.

In the example shown in FIG. 1. negative difference values (d<0) are linearly amplified. The amplification factor is 1.6̅ in the example shown. The difference value d=−300 yields for example the amplified difference value −300·1.6=−500.

Positive difference values above an upper positive difference value dₘₐₓ are likewise linearly amplified; in this range of difference values as well, the amplification factor in the example shown is 1.6̅. The difference value d=900 yields for example the amplified difference value 900·1.6̅=1500.

In a range between the difference value zero (d=0) and the upper positive difference value dₘₐₓ, the difference values are non-linearly amplified. The amplification factor is not constant in this range; it depends on the respective difference value. In the example shown, the non-linear amplification is lower than the linear amplification above the upper positive difference value dₘₐₓ and in the range of the negative difference values. The dashed line indicates the linear amplification.

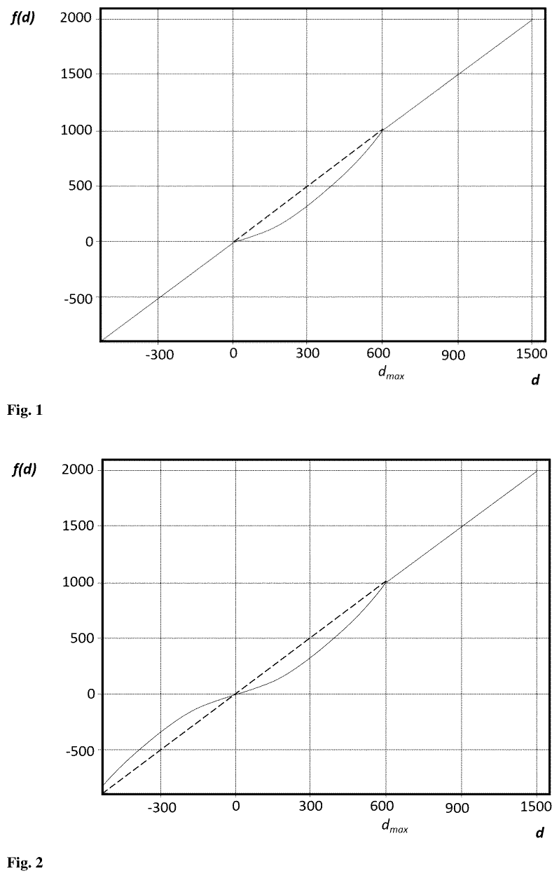

FIG. 2 shows in schematic form a further example of a non-linear transformation according to the present disclosure.

Difference values d are transformed by means of a transformation function ƒ(d). In FIG. 2, the transformed difference values are plotted as a function ƒ(d) of the difference values d in a graphical representation in a Cartesian coordinate system.

In the example shown in FIG. 2, positive difference values above an upper positive difference value dₘₐₓ are linearly amplified. In this range of difference values, the amplification factor is 1.6 in the example shown. The difference value d=900 yields for example the amplified difference value 900·1.6̅=1500.

In a range between the difference value zero (d=0) and the upper positive difference value dₘₐₓ, the difference values are non-linearly amplified. The amplification factor is not constant in this range; it depends on the respective difference value. In the example shown, the non-linear amplification is lower than the linear amplification above the upper positive difference value dₘₐₓ. The dashed line indicates the linear amplification. A linear amplification of the difference value d=300 would yield an amplified difference value of 300·1.6̅=500, but the amplified difference value is lower than 500 (it is about 322).

In the example shown, negative difference values (d<0) are likewise non-linearly amplified. In the range of negative difference values too, the amplification is lower than the linear amplification (shown by the dashed line). A linear amplification of the difference value d=−300 would yield an amplified difference value of −300·1.6̅=−500, but the amplified difference value is lower than −500 (note that the term “amplification” as described above relates to the absolute value. i.e. in the example shown. a value of about −322 would yield: |−322|<|−500|).

In the example shown: ƒ⁽⁻⁾(d⁽⁻⁾)=−ƒ⁽⁺⁾(|d⁽⁻⁾|), i.e. the magnitudes formed from negative difference values are amplified in the same way as positive difference values.

The third representation non-linearly transformed over at least a portion of the difference values is also referred to as transformed third representation in this description. The fourth representation may be the transformed third representation.

In one embodiment of the present disclosure, the generation of the fourth representation further comprises multiplication of the third representation and/or the transformed third representation by a frequency-dependent weight function in frequency space.

The multiplication of the third representation and/or the transformed third representation by a frequency-dependent weight function in frequency space can be considered to be a filter which, depending on the weight function, leads to an amplification and/or attenuation of frequencies. For example, if low frequencies are weighted higher than high frequencies, a low-pass filter is present. For example, if high frequencies are weighted higher than low frequencies, a high-pass filter is present.

The multiplication of the third representation and/or the transformed third representation by a frequency-dependent weight function is carried out in frequency space. If the third representation and/or the transformed third representation are not present in frequency space, but in real space for example, then they can be converted into the corresponding frequency-space representation by a transformation, for example a Fourier transform.

The multiplication normally comprises multiplication of each amplitude value/phase value of each frequency of the frequency-space representation by a frequency-dependent weighting factor.

Preferably, the weighting factors decrease with increasing frequency. In other words: Preferably, low frequencies are multiplied by a higher weighting factor than high frequencies. Preferably, the greater the particular weighting factor, the lower the frequency.

Contrast information is represented in a frequency-space depiction by low frequencies, while the higher frequencies represent information about fine structures. Higher weighting of low frequencies thus means that a higher weighting will be given to frequencies making a higher contribution to contrast than to those making a smaller contribution. Image noise is typically evenly distributed in the frequency depiction. The frequency-dependent weight function has the effect of a filter. The filter increases the signal-to-noise ratio by reducing the spectral noise density for high frequencies.

Preferred weight functions are Gaussian function, Hann function (also referred to as the Hann window) and Poisson function (Poisson window).

Examples of weight other functions can be found for example at https://de.wikipedia.org/wiki/Fensterfunktion#Beispiele_von_Fensterfunktionen; F. J. Harris et al.: On the Use of Windows for Harmonic Analysis with the Discrete Fourier Transform, Proceedings of the IEEE, vol. 66. No. 1. 1978; https://docs.scipy.org/doc/scipy/reference/signal.windows.html; K. M. M. Prabhu: Window Functions and Their Applications in Signal Processing, CRC Press, 2014, 978-1-4665-1583-3).

Frequency-dependent weighting is also described in WO2024/052156A1. The weight functions disclosed in WO2024/052156A1 may also be used to filter the representations described herein.

As an alternative or in addition to frequency-dependent weighting, other/further forms of filtering may also be performed.

On the basis of the fourth representation, a synthetic contrast-enhanced representation of the examination region of the examination object is generated. The generation of the synthetic contrast-enhanced representation comprises addition of the fourth representation to the first representation or to the second representation. The addition may for example be carried out in real space or in frequency space. If the addition is carried out in real space, the values assigned to the image elements of the fourth representation are added image element by image element to the colour values or grey values of the corresponding image elements of the first representation or the second representation. Corresponding image elements are those that represent the same subregion of the examination region.

It is possible to normalize the colour values or grey values of the synthetic contrast-enhanced representation.

The synthetic contrast-enhanced representation represents the examination region of the examination object after administration of a third amount of the contrast agent. The third amount is normally greater than the first amount and the second amount.

In one embodiment of the present disclosure, the first amount and the second amount are less than the standard amount and the third amount is equal to the standard amount or greater than the standard amount.

In one embodiment, the first amount is less than the standard amount (e.g. zero), the second amount corresponds to the standard amount, and the third amount is greater than the standard amount.

If the synthetic contrast-enhanced representation is not present in a real-space depiction, but in a frequency-space depiction for example, it can be converted into a real-space depiction by a transformation, for example an inverse Fourier transform.

The synthetic contrast-enhanced representation can be output, i.e. displayed on a monitor and/or printed by means of a printer, and/or stored in a data memory. The synthetic contrast-enhanced representation can be transmitted to a separate computer system.

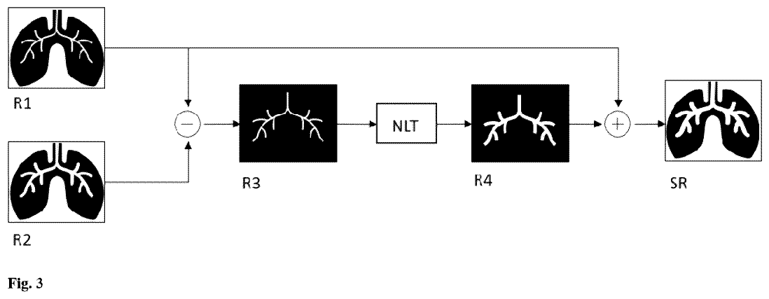

FIG. 3 shows by way of example and in schematic form the generation of a synthetic contrast-enhanced representation.

The generation of the synthetic contrast-enhanced representation SR is based on a first representation R1 and a second representation R2. In the example shown in FIG. 3, the first representation R1 and the second representation R2 are radiological images of a human. The examination region comprises the lungs of the human.

The first representation R1 represents the examination region without contrast agent. The second representation R2 represents the examination region after administration of an amount of a contrast agent.

On the basis of the first representation R1 and the second representation R2, a third representation R3 is generated. The generation of the third representation R3 comprises subtraction of the first representation R1 from the second representation R2. In the example shown in FIG. 3, the subtraction is carried out in real space. As described, it is possible to also carry out the subtraction in a different space, for example in frequency space. The subtraction comprises subtraction of the grey values of corresponding image elements from one another. Corresponding image elements are those that represent the same subregion of the examination region.

The third representation R3 also comprises a multiplicity of image elements. Each image element is assigned a difference value. The difference values are yielded by the subtraction of the grey values of the image elements of the first representation R1 from the grey values of the corresponding image elements of the second representation R2.

In a further step, the difference values are transformed. Here, there is at least one range of difference values that is non-linearly transformed. The non-linear transformation of at least a portion of the difference values is represented in FIG. 3 by the box labelled NLT. Examples of non-linear transformation of at least a portion of difference values are shown in FIGS. 1 and 2. The non-linear transformation of at least a portion of the difference values is done in real space. The result is a fourth representation R4.

In a further step, the fourth representation R4 is added to the first representation R1. It is also possible to add the fourth representation R4 to the second representation. The addition of the fourth representation R4 to the first representation R1 (or to the second representation R2) may be carried out in real space or in frequency space. In the example shown in FIG. 3, it is carried out in real space. The result is a synthetic contrast-enhanced representation SR of the examination region of the examination object. The synthetic contrast-enhanced representation SR represents the examination region of the examination object after administration of an amount of the contrast agent that is greater than in the case of the second representation R2.

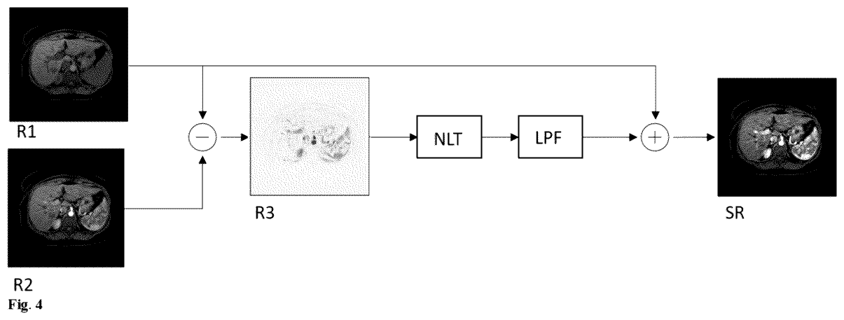

FIG. 4 shows by way of example and in schematic form a further embodiment of the generation of a synthetic contrast-enhanced representation.

The generation of the synthetic contrast-enhanced representation SR is based on a first representation R1 and a second representation R2. In the example shown in FIG. 4, the first representation R1 and the second representation R2 are MRI images of a human. The examination region comprises a section through the abdomen; what can be identified is, inter alia, a liver.

The first representation RI represents the examination region without contrast agent. The second representation R2 represents the examination region after administration of an amount of a hepatobiliary MRI contrast agent.

On the basis of the first representation R1 and the second representation R2, a third representation R3 is generated. The generation of the third representation R3 comprises subtraction of the first representation R1 from the second representation R2. In the example shown in FIG. 4, the subtraction is carried out in real space. As described, it is possible to also carry out the subtraction in a different space, for example in frequency space. The subtraction comprises subtraction of the grey values of corresponding image elements from one another. Corresponding image elements are those that represent the same subregion of the examination region.

The third representation R3 also comprises a multiplicity of image elements. Each image element is assigned a difference value. The difference values are yielded by the subtraction of the grey values of the image elements of the first representation R1 from the grey values of the corresponding image elements of the second representation R2.

In a further step, the difference values are transformed. Here, there is at least one range of difference values that is non-linearly transformed. The non-linear transformation of at least a portion of the difference values is represented in FIG. 4 by the box labelled NLT. Examples of non-linear transformation of at least a portion of difference values are shown in FIGS. 1 and 2. The non-linear transformation of at least a portion of the difference values is done in real space.

The non-linear transformation NLT of at least a portion of the difference values is followed by a low-pass filter LPF. With this low-pass filter LPF, the amplitude and/or phase values of a frequency-space depiction of the at least partly non-linearly transformed representation are multiplied by a frequency-dependent weight function in which low frequencies are weighted higher than high frequencies. In FIG. 4, the low-pass filter is represented by the box labelled LPF.

In the example shown in FIG. 4, the at least partial non-linear transformation NLT takes place first, followed by the low-pass filtering LPF. It is also possible that the low-pass filter (and/or a different filter and/or a further filter) is applied to the third representation R3 before the filtering result is at least partly non-linearly transformed.

In a further step, the result of the at least partial non-linear transformation and low-pass filtering is added to the first representation R1. This step may be carried out in real space or in frequency space. In the example shown in FIG. 4, it is carried out in real space. The result is a synthetic contrast-enhanced representation SR of the examination region of the examination object. The synthetic contrast-enhanced representation SR represents the examination region of the examination object after administration of an amount of the hepatobiliary contrast agent that is greater than in the case of the second representation R2.

Thus the subjects of the present disclosure allow the generation of a synthetic contrast-enhanced representation of an examination region of an examination object that represents the examination region of the examination object after administration of an amount of a contrast agent that is greater than the amount actually administered. As a result, either contrast agents can be saved, or synthetic contrast-enhanced representations can be generated that represent the examination region of the examination object after administration of an amount of a contrast agent that is greater than the amount that would normally be administered to an examination object.

A synthetic contrast-enhanced representation generated according to the present disclosure has a greater contrast-to-noise ratio than synthetic contrast-enhanced representations generated according to the methods disclosed in WO2024/100233A1 and WO2024/052156A1. This is shown by way of example later in the description.

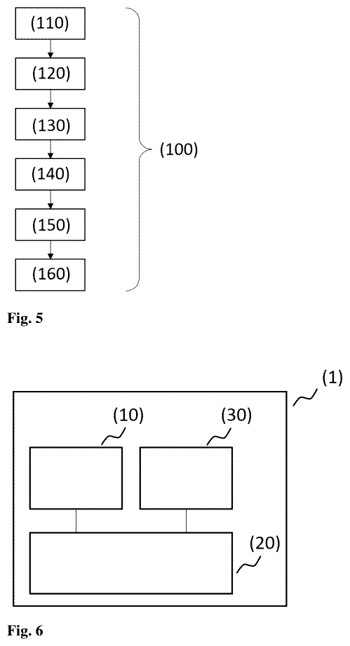

FIG. 5 shows by way of example and in schematic form a method for generating a synthetic contrast-enhanced representation in the form of a flow chart.

The method (100) comprises the steps of:
(110) providing a first representation, where the first representation represents an examination region of an examination object without contrast agent or after administration of a first amount of a contrast agent,
(120) providing a second representation, where the second representation represents the examination region of the examination object after administration of a second amount of the contrast agent.
(130) generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
(140) generating a fourth representation of the examination region of the examination object, where the generation of the fourth representation comprises non-linear transformation of at least a portion of the difference values of the third representation,
(150) generating a synthetic contrast-enhanced representation of the examination region of the examination object, where the generation of the synthetic contrast-enhanced representation comprises addition of the fourth representation to the first representation or to the second representation.
(160) outputting and/or storing the synthetic contrast-enhanced representation of the examination region of the examination object and/or transmitting the synthetic contrast-enhanced representation to a separate computer system.

FIG. 6 shows by way of example and in schematic form a computer system according to the present disclosure.

A “computer system” is an electronic data processing system that processes data by means of programmable calculation rules. Such a system typically comprises a “computer”, which is the unit that includes a processor for carrying out logic operations, and peripherals.

In computer technology. “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (1) shown in FIG. 6 comprises a receiving unit (10), a control and calculation unit (20) and an output unit (30).

The control and calculation unit (20) serves for control of the computer system (1). for coordination of the data flows between the units of the computer system (1), and for the performance of calculations.

The control and calculation unit (20) is configured:
to provide a first representation and/or cause the receiving unit (10) to receive a first representation. where the first representation represents an examination region of an examination object without contrast agent or after administration of a first amount of a contrast agent.
to provide a second representation and/or to cause the receiving unit (10) to receive a second representation, where the second representation represents the examination region of the examination object after administration of a second amount of the contrast agent.
to generate a third representation of the examination region of the examination object on the basis of the first representation and the second representation, where the generation of the third representation comprises subtraction of the first representation from the second representation. where the third representation comprises a multiplicity of image elements, where each image element is assigned at least one difference value.
to generate a fourth representation of the examination region of the examination object, where the generation of the fourth representation comprises non-linear transformation of at least a portion of the difference values of the third representation.
to generate a synthetic contrast-enhanced representation of the examination region of the examination object. where the generation of the synthetic contrast-enhanced representation comprises addition of the fourth representation to the first representation or to the second representation.
to store the synthetic contrast-enhanced representation of the examination region of the examination object or to cause the output unit (30) to output the synthetic contrast-enhanced representation and/or to transmit the synthetic contrast-enhanced representation to a separate computer system.

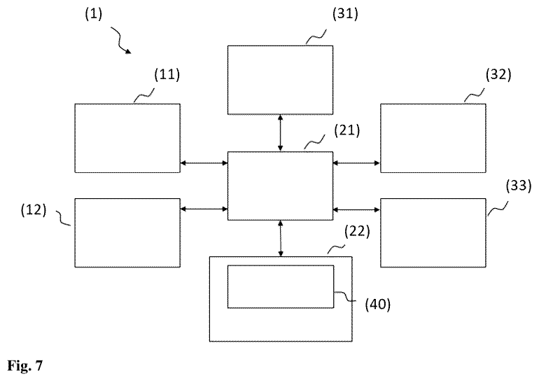

FIG. 7 shows by way of example and in schematic form a further embodiment of the computer system of the present disclosure. The computer system (1) comprises a processing unit (21) connected to a memory (22). The processing unit (21) and the memory (22) form a control and calculation unit, as shown in FIG. 6.

The processing unit (21) may comprise one or more processors alone or in combination with one or more memories. The processing unit (21) can be customary computer hardware that is able to process information such as e.g. digital image recordings, computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of the same or of a different computer system.

The memory (22) may be customary computer hardware that is able to store information such as digital images (for example representations of the examination region), data, computer programs and/or other digital information either temporarily and/or permanently. The memory (22) may comprise a volatile and/or nonvolatile memory and may be nonremovable or removable. Examples of suitable memories are RAM (random access memory). ROM (read-only memory), a hard disk, a flash memory, an exchangeable computer floppy disk, an optical disc. a magnetic tape or a combination of the aforementioned. Optical discs can include compact discs with read-only memory (CD-ROM), compact discs with read/write function (CD-R/W), DVDs. Blu-ray discs and the like.

The processing unit (21) may be connected not just to the memory (22), but also to one or more interfaces (11, 12, 31, 32, 33) in order to display, transmit and/or receive information. The interfaces may comprise one or more communication interfaces (11, 32, 33) and/or one or more user interfaces (12, 31). The one or more communication interfaces may be configured to send and/or receive information, for example to and/or from an MRI scanner, a CT scanner, an ultrasound camera, other computer systems. networks, data memories or the like. The one or more communication interfaces can be configured to transmit and/or receive information via physical (wired) and/or wireless communication connections. The one or more communication interfaces can comprise one or more interfaces for connection to a network, for example using technologies such as mobile phone. Wi-Fi, satellite, cable, DSL, optical fibre and/or the like. In some examples, the one or more communication interfaces can comprise one or more close-range communication interfaces configured to connect devices with close-range communication technologies such as NFC, RFID, Bluetooth, Bluetooth LE, ZigBee, infrared (e.g. IrDA) or the like.

The user interfaces may include a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image or video recording device (for example a camera), a keyboard or a keypad, a joystick, a touch-sensitive surface (separate from a touchscreen or integrated therein) or the like. In some examples, the user interfaces can contain an automatic identification and data capture technology (AIDC) for machine-readable information. This can include barcodes, radiofrequency identification (RFID). magnetic strips. optical character recognition (OCR), integrated circuit cards (ICC) and the like. The user interfaces can furthermore comprise one or more interfaces for communication with peripherals such as printers and the like.

One or more computer programs (40) may be stored in the memory (22) and executed by the processing unit (21), which is thereby programmed to perform the functions described in this description. The retrieving. loading and execution of instructions of the computer program (40) may take place sequentially, such that an instruction is respectively retrieved, loaded and executed. However, the retrieving, loading and/or execution may also take place in parallel.

The computer system of the present disclosure may be designed as a laptop, notebook, netbook and/or tablet PC; it may also be a component of an MRI scanner, a CT scanner, a PET scanner or an ultrasound diagnostic device.

The present invention also provides a computer program product. Such a computer program product includes a non-volatile data carrier, for example a CD, a DVD, a USB stick or another data storage medium. Stored on the data carrier is a computer program. The computer program can be loaded into a working memory of a computer system (more particularly into a working memory of a computer system of the present disclosure), where it causes the computer system to execute the following steps:
providing a first representation, where the first representation represents an examination region of an examination object without contrast agent or after administration of a first amount of a contrast agent,
providing a second representation, where the second representation represents the examination region of the examination object after administration of a second amount of the contrast agent,
generating a third representation of the examination region of the examination object on the basis of the first representation and the second representation, where the generation of the third representation comprises subtraction of the first representation from the second representation,
where the third representation comprises a multiplicity of image elements, where each image element is assigned at least one difference value,
generating a fourth representation of the examination region of the examination object, where the generation of the fourth representation comprises non-linear transformation of at least a portion of the difference values of the third representation,
generating a synthetic contrast-enhanced representation of the examination region of the examination object, where the generation of the synthetic contrast-enhanced representation comprises addition of the fourth representation to the first representation or to the second representation,
outputting and/or storing the synthetic contrast-enhanced representation of the examination region of the examination object and/or transmitting the synthetic contrast-enhanced representation to a separate computer system.

The computer program can also be available for purchase as a computer program product as a download, for example via a webpage and/or an app store.

The computer program product can also be marketed in combination (in a set) with the contrast agent. Such a set is also referred to as a kit. Such a kit comprises the contrast agent and the computer program product. It is also possible for such a kit to comprise the contrast agent and means allowing a purchaser to obtain the computer program, for example to download it from a webpage. These means may include a link, i.e. an address of the webpage on which the computer program can be obtained, for example from which the computer program can be downloaded to a computer system connected to the internet. These means may include a code (for example an alphanumeric string or a QR code, or a DataMatrix code or a barcode or another optically and/or electronically readable code) that gives the purchaser access to the computer program. Such a link and/or code may for example be printed on a packaging of the contrast agent and/or on a package leaflet of the contrast agent. A kit is thus a combination product comprising a contrast agent and a computer program (e.g. in the form of access to the computer program or in the form of executable program code on a data carrier) that are available for purchase together.

The subjects of the present disclosure can be used for various purposes. A few examples of use will be described below without any intention of limiting the disclosure to said examples of use.

A first example of use concerns magnetic resonance imaging examinations for differentiating intraaxial tumours such as intracerebral metastases and malignant gliomas. The infiltrative growth of these tumours makes it difficult to differentiate exactly between tumour and healthy tissue. Determining the extent of a tumour is however crucial for surgical removal. Distinguishing between tumours and healthy tissue is facilitated by administration of an extracellular MRI contrast agent; after intravenous administration of a standard dose of 0.1 mmol/kg body weight of the extracellular MRI contrast agent gadobutrol, intraaxial tumours can be differentiated much more readily. At higher doses, the contrast between lesion and healthy brain tissue is increased further; the detection rate of brain metastases increases linearly with the dose of the contrast agent (see for example M. Hartmann et al.: Does the administration of a high dose of a paramagnetic contrast medium (Gadovist) improve the diagnostic value of magnetic resonance tomography in glioblastomas? doi: 10.1055/s-2007-1015623).

A single triple dose or a second subsequent dose may be administered here up to a total dose of 0.3 mmol/kg body weight. This exposes the patient and the environment to additional gadolinium and in the case of a second scan, incurs additional costs.

The subjects of the present disclosure can be used to avoid a dose of contrast agent that exceeds the standard amount. A first MRI image can be generated without contrast agent or with less than the standard amount and a second MRI image can be generated with the standard amount. On the basis of these generated MRI images, it is possible, as described in this disclosure, to generate a synthetic MRI image in which the contrast between lesions and healthy tissue can be varied within wide limits by an at least partial non-linear amplification. This makes it possible to achieve contrasts that are otherwise achievable only by administering an amount of contrast agent larger than the standard amount.

Another example of use concerns the reduction of the amount of MRI contrast agent in a magnetic resonance imaging examination. Gadolinium-containing contrast agents such as gadobutrol are used for a multitude of examinations. They are used for contrast enhancement in skull examinations, spine examinations, breast examinations or other examinations. In the central nervous system, gadobutrol highlights regions where the blood-brain barrier is impaired and/or vessels are abnormal. In breast tissue, gadobutrol visualizes the presence and extent of a malignant breast disease. Gadobutrol is also used in contrast-enhanced magnetic resonance angiography for diagnosing strokes, for detecting tumour blood perfusion and for detecting focal cerebral ischaemia.

Owing to the increasing impact on the environment, to the cost burden falling on health care systems and to concerns about acute side effects and possible long-term health risks, especially in the case of repeated and long-term exposure, a reduction in the dose of gadolinium-containing contrast agents is desired. This can be achieved by the subjects of the present disclosure.

A first MRI image without contrast agent and a second MRI image with an amount of contrast agent less than the standard amount can be generated. On the basis of these generated MRI images, it is possible, as described in this disclosure, to generate a synthetic MRI image in which the contrast can be varied within wide limits by the at least partial non-linear amplification. This makes it possible with less than the standard amount of contrast agent to achieve the same contrast as is obtained after administration of the standard amount.

Another example of use concerns the detection, identification and/or characterization of lesions in the liver with the aid of a hepatobiliary contrast agent such as Primovist®.

Primovist® is administered intravenously (i.v.) at a standard dose of 0.025 mmol/kg body weight. This standard dose is lower than the standard dose of 0.1 mmol/kg body weight in the case of extracellular MRI contrast agents. Unlike in contrast-enhanced MRI with extracellular gadolinium-containing contrast agents. Primovist® permits dynamic multiphase T1w imaging. However, the lower dose of Primovist® and the observation of transient motion artefacts that can occur shortly after intravenous administration means that contrast enhancement with Primovist® in the arterial phase is perceived by radiologists as poorer than contrast enhancement with extracellular MRI contrast agents. The assessment of contrast enhancement in the arterial phase and of the vascularity of focal liver lesions is however of critical importance for accurate characterization of the lesion.

With the aid of the present invention it is possible to increase contrast, particularly in the arterial phase. without the need to administer a higher dose.

A first MRI image without contrast agent and a second MRI image during the arterial phase after administering an amount of a contrast agent that corresponds to the standard amount can be generated. On the basis of these generated MRI images, it is possible, as described in this disclosure, to generate a synthetic MRI image in which the contrast in the arterial phase can be varied within wide limits by the at least partial non-linear amplification. This makes it possible to achieve contrasts that are otherwise achievable only by administering an amount of contrast agent larger than the standard amount.

Another example of use concerns the use of MRI contrast agents in computed tomography examinations.

In a CT examination, MRI contrast agents usually have a lower contrast-enhancing effect than CT contrast agents. However, it can be advantageous to employ an MRI contrast agent in a CT examination. An example is a minimally invasive intervention in the liver of a patient, where a surgeon is monitoring the procedure by means of a CT scanner. Computed tomography (CT) has the advantage over magnetic resonance imaging that more major surgical interventions are possible in the examination region while generating CT images of an examination region of an examination object. On the other hand, there are only few surgical instruments and surgical devices that are MRI-compatible. Moreover, access to the patient is restricted by the magnets used in MRI. Thus, while performing an operation in the examination region, a surgeon will be able visualize the examination region by CT and to follow the operation on a monitor.

For example, if a surgeon wishes to perform a procedure in a patient's liver in order for example to carry out a biopsy on a liver lesion or to remove a tumour, the contrast between a liver lesion or tumour and healthy liver tissue will not be as pronounced in a CT image of the liver as it is in an MRI image after administration of a hepatobiliary contrast agent. There are currently no known and/or authorized CT-specific hepatobiliary contrast agents in CT. The use of an MRI contrast agent, more particularly a hepatobiliary MRI contrast agent, in computed tomography thus combines the possibility of differentiating between healthy and diseased liver tissue and the possibility of carrying out an operation with simultaneous visualization of the liver.

The comparatively low contrast enhancement achieved by the MRI contrast agent can be increased with the aid of the subjects of the present disclosure without the need to administer a dose higher than the standard dose.

A first CT image without MRI contrast agent and a second CT image after administering an amount of an MRI contrast agent that corresponds to the standard amount can be generated. On the basis of these generated CT images, it is possible, as described in this disclosure, to generate a synthetic CT image in which the contrast caused by the MRI contrast agent can be varied within wide limits by the at least partial non-linear amplification. This makes it possible to achieve contrasts that are otherwise achievable only by administering an amount of MRI contrast agent larger than the standard amount.

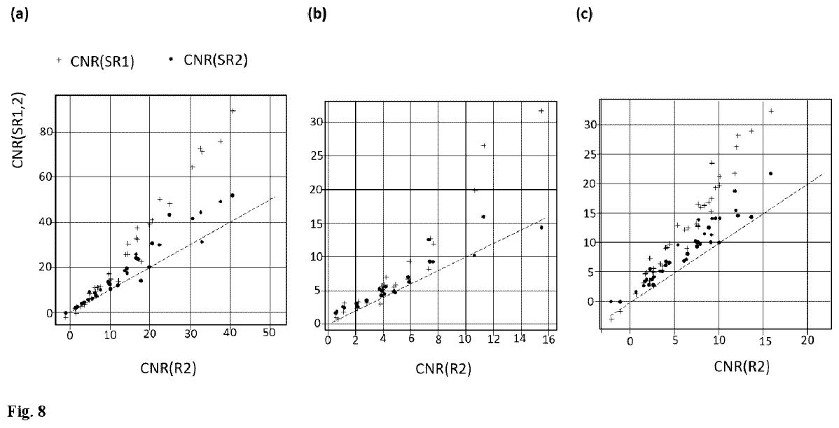

FIG. 8 compares the contrast-to-noise ratios of synthetic contrast-enhanced representations that were generated by different methods.

What were generated for each human of a multiplicity of humans were MRI images of an examination region of the human before and after administration of a hepatobiliary contrast agent. The examination region comprises the liver of the human. A first MRI image represented the examination region in the native phase (before administration of the contrast agent); a second MRI image represented the examination region in the arterial phase (after administration of the contrast agent).

On the basis of the first MRI image and the second MRI image, a first synthetic contrast-enhanced MRI image was generated as described in this disclosure. The upper 0.99 quantile was linearly amplified with an amplification factor of 1.8 and the lower 0.99 quantile was non-linearly amplified according to formulae (2a) and (2b) with the following values:

$${β}^{( + )} = {β}^{( - )} = 1.8$$

$${δ}^{( + )} = {δ}^{( - )} = 0$$

$${γ}^{( + )} = {γ}^{( - )} = 1.6$$

In other words, 99% of the difference values were non-linearly amplified, whereas 1% of the difference values were linearly amplified.

In addition, the amplification was also followed by application of a low-pass filter in the form of a Gaussian curve with a standard deviation of 0.36.

For comparison, a second synthetic contrast-enhanced MRI image was generated for each human of the multiplicity of humans, said MRI image differing from the first synthetic contrast-enhanced MRI image in that all difference values were linearly amplified by a constant amplification factor of 1.8. The second synthetic contrast-enhanced MRI image thus does not have non-linear amplification.

The contrast-to-noise ratio CNR of the second contrast-enhanced MRI image and of the first and second synthetic contrast-enhanced MRI image was calculated according to the following formula:

$$CNR = \frac{{I}_{T} - {I}_{L}}{{STD}_{L}} \qquad ( 3 )$$

Here, I_{T} is the average (arithmetic mean) signal intensity (e.g. in the form of a grey value) of a defined tissue (an aorta, a liver lesion and a portal vein in the present example), I_{L} is the average (arithmetic mean) signal intensity of healthy liver cells, and STD_{L} is the standard deviation of the signal intensities of healthy liver cells (reference).

In FIG. 8, the contrast-to-noise ratios of the synthetic contrast-enhanced MRI images are plotted against the contrast-to-noise ratio of the second MRI image for all the humans examined.

FIG. 8(a) shows the plot for the aorta, FIG. 8(b) shows the plot for a liver lesion, and FIG. 8(c) shows the plot for the portal vein.

Each cross indicates a first synthetic contrast-enhanced MRI image, i.e. an MRI image in which non-linear amplification has been performed at least in part. Each circle indicate a second synthetic contrast-enhanced MRI image, i.e. an MRI image in which linear amplification has been performed over the full range of difference values.

The dashed lines indicate the same contrast-to-noise ratios.

It can be seen that both the first synthetic contrast-enhanced MRI images and the second synthetic contrast-enhanced MRI images have higher contrast-to-noise ratios than the non-synthetically enhanced MRI images; both the crosses and the circles lie within wide ranges above the dashed lines. However, the first synthetic contrast-enhanced MRI images have higher contrast-to-noise ratios than the second synthetic contrast-enhanced MRI images.

1. Computer-implemented method comprising:
providing a first representation, where the first representation represents an examination region of an examination object without contrast agent or after administration of a first amount of a contrast agent,
providing a second representation, where the second representation represents the examination region of the examination object after administration of a second amount of the contrast agent,
generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
generating a fourth representation of the examination region of the examination object, where the generation of the fourth representation comprises non-linear transformation of at least a portion of the difference values of the third representation,
generating a synthetic contrast-enhanced representation of the examination region of the examination object, where the generation of the synthetic contrast-enhanced representation comprises addition of the fourth representation to the first representation or to the second representation,
outputting and/or storing the synthetic contrast-enhanced representation of the examination region of the examination object and/or transmitting the synthetic contrast-enhanced representation to a separate computer system.

2. Method according to claim 1, wherein the non-linear transformation is a non-linear amplification and/or a non-linear attenuation, non-linear amplification meaning an increase in magnitudes of difference values and non-linear attenuation meaning a decrease in magnitudes of difference values.

3. Method according to claim 1 or 2, wherein the non-linear transformation comprises an increase and/or decrease in magnitudes of difference values, the increase and/or decrease depending on the size of the difference values.

4. Method according to any of claims 1 to 3, wherein the non-linear transformation comprises a non-linear amplification of at least a portion of the positive difference values of the third representation.

5. Method according to any of claims 1 to 4, wherein negative difference values and positive difference values are amplified, at least a portion of the positive difference values being non-linearly amplified.

6. Method according to any of claims 1 to 5, wherein there is an upper positive difference value, and difference values above the upper positive difference value are linearly amplified, whereas difference values between the difference value zero and the upper positive difference value are non-linearly amplified.

7. Method according to claim 6, wherein the non-linear amplification is lower than the linear amplification.

8. Method according to any of claims 1 to 7, wherein all positive difference values are amplified by a factor, the factor being zero for the difference value zero and assuming a maximum value for an upper positive difference value and assuming a minimum value for a difference value between the difference value zero and the upper positive difference value.

9. Method according to any of claims 1 to 8, wherein the difference values of the upper p-quantile of the positive difference values are linearly amplified, whereas the difference values of the lower p-quantile of the positive difference values are non-linearly amplified, p being a real number that assumes a value in the range from 0.5 to 0.99 or 0.6 to 0.99 or 0.7 to 0.99 or 0.8 to 0.99 or 0.9 to 0.99 or 0.95 to 0.99.

10. Method according to any of claims 1 to 9, wherein the difference values within a range of difference values are transformed by means of a transformation function, the transformation function comprising a power function of the respective difference value.

11. Method according to any of claims 1 to 10, wherein the difference values within a range of difference values are transformed by means of a transformation function, the transformation function being a continuous and continuously differentiable function.

12. Method according to any of claims 1 to 11, wherein at least a portion of the difference values are transformed by means of a transformation function, the transformation function comprising a transformation function ƒ⁽⁺⁾ that transforms positive difference values and a transformation function ƒ⁽⁻⁾ that transforms negative difference values, where:
$${f}^{( - )} ( {d}^{( - )} ) = - {f}^{( + )} ( \vert {d}^{( - )} \vert )$$
where d⁽⁻⁾ is a negative difference value and |d⁽⁻⁾| is the magnitude of the negative difference value.

13. Method according to any of claims 1 to 12, wherein difference values within a range of difference values are transformed by means of a transformation function, the transformation function having the following form:
$${f}^{( + )} ( {d}^{( + )} ) = {β}^{( + )} \cdot {d}^{( + )} \cdot {( \frac{{d}^{( + )}}{{d}_{max}^{( + )}} )}^{{γ}^{( + )}} + {δ}^{( + )}$$
$${f}^{( - )} ( {d}^{( - )} ) = {β}^{( - )} \cdot {d}^{( - )} \cdot {( \frac{\vert {d}^{( - )} \vert}{{\vert {d}^{( - )} \vert}_{max}} )}^{{γ}^{( - )}} + {δ}^{( - )}$$
where ƒ⁽⁺⁾(d⁽⁺⁾) is a transformation function that transforms positive difference values,
where ƒ⁽⁻⁾(d⁽⁻⁾) is a transformation function that transforms negative difference values,
where β⁽⁺⁾ and β⁽⁻⁾ are factors that each independently assume a value greater than 1,
where d⁽⁺⁾ is a positive difference value that is transformed,
where
$${d}_{max}^{( + )}$$
is the maximum positive difference value that occurs in the range of difference values,
where d⁽⁻⁾ is a negative difference value that is transformed,
where |d⁽⁻⁾| is the magnitude of the negative difference value d⁽⁻⁾,
where |d⁽⁻⁾|ₘₐₓ is the maximum magnitude that occurs in the range of negative difference values,
where γ⁽⁺⁾ and γ⁽⁻⁾ are exponents that each independently assume a positive real value,
where δ⁽⁺⁾ and δ⁽⁻⁾ each independently assume a value of zero or a positive or negative real value.

14. Method according to claim 13, wherein γ⁽⁺⁾ and γ⁽⁻⁾ assume a value in the range from 1.1 to 3, or a value in the range from 1.2 to 2.9, or a value in the range from 1.3 to 2.8, or a value in the range from 1.4 to 2.7, or a value in the range from 1.5 to 2.6.

15. Method according to any of claims 1 to 14, wherein the examination object is a living being, and/or a mammal and/or a human.

16. Method according to any of claims 1 to 15, wherein the examination region is a liver, kidney, heart, lung, brain, stomach, bladder, pancreas, prostate, breast, intestine or a part thereof or another part of the body of a mammal and/or human or includes at least a part thereof.

17. Method according to any of claims 1 to 16,
wherein the synthetic contrast-enhanced representation represents the examination region after administration of a third amount of the contrast agent,
where the first amount is zero or the first amount is not equal to zero and is less than the standard amount,
where the second amount is less than the standard amount, and
where the third amount is equal to or greater than the standard amount.

18. Method according to any of claims 1 to 17,
wherein the synthetic contrast-enhanced representation represents the examination region after administration of a third amount of the contrast agent,
where the first amount is zero or the first amount is not equal to zero and is less than the standard amount,
where the second amount is equal to the standard amount, and
where the third amount is greater than the standard amount.

19. Method according to any of claims 1 to 18, wherein the first representation and the second representation are the result of a radiological examination on the examination object.

20. Method according to any of claims 1 to 19, wherein
the first representation and the second representation are a result of a magnetic resonance imaging examination and/or have been generated from magnetic resonance images, or
the first representation and the second representation are a result of a computed tomography examination and/or have been generated from computed tomography images.

21. Method according to any of claims 1 to 20, wherein the contrast agent is an MRI contrast agent and/or a hepatobiliary contrast agent.

22. Computer system (1) comprising
a receiving unit (10),
a control and calculation unit (20) and
an output unit (30),
wherein the control and calculation unit (20) is configured
to provide a first representation and/or cause the receiving unit (10) to receive a first representation, where the first representation represents an examination region of an examination object without contrast agent or after administration of a first amount of a contrast agent,
to provide a second representation and/or to cause the receiving unit (10) to receive a second representation, where the second representation represents the examination region of the examination object after administration of a second amount of the contrast agent,
to generate a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
to generate a fourth representation of the examination region of the examination object, where the generation of the fourth representation comprises non-linear transformation of at least a portion of the difference values of the third representation,
to generate a synthetic contrast-enhanced representation of the examination region of the examination object, where the generation of the synthetic contrast-enhanced representation comprises addition of the fourth representation to the first representation or to the second representation,
to store the synthetic contrast-enhanced representation of the examination region of the examination object or to cause the output unit (30) to output the synthetic contrast-enhanced representation and/or to transmit the synthetic contrast-enhanced representation to a separate computer system.

23. Computer program product comprising a data carrier on which there is stored a computer program (40) that can be loaded into a working memory (22) of a computer system (1), where it causes the computer system (1) to execute the following steps:
providing a first representation, where the first representation represents an examination region of an examination object without contrast agent or after administration of a first amount of a contrast agent,
providing a second representation, where the second representation represents the examination region of the examination object after administration of a second amount of the contrast agent.
generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
generating a fourth representation of the examination region of the examination object, where the generation of the fourth representation comprises non-linear transformation of at least a portion of the difference values of the third representation,
generating a synthetic contrast-enhanced representation of the examination region of the examination object, where the generation of the synthetic contrast-enhanced representation comprises addition of the fourth representation to the first representation or to the second representation,
outputting and/or storing the synthetic contrast-enhanced representation of the examination region of the examination object and/or transmitting the synthetic contrast-enhanced representation to a separate computer system.

24. Use of a contrast agent in a radiological examination method comprising the steps of:
generating a first representation, where the first representation represents an examination region of an examination object without contrast agent or after administration of a first amount of the contrast agent,
generating a second representation, where the second representation represents the examination region of the examination object after administration of a second amount of the contrast agent,
generating a third representation of the examination region of the examination object on the basis of the first representation and the second representation, where the generation of the third representation comprises subtraction of the first representation from the second representation, where the third representation comprises a multiplicity of image elements, where each image element is assigned at least one difference value,
generating a fourth representation of the examination region of the examination object, where the generation of the fourth representation comprises non-linear transformation of at least a portion of the difference values of the third representation,
generating a synthetic contrast-enhanced representation of the examination region of the examination object, where the generation of the synthetic contrast-enhanced representation comprises addition of the fourth representation to the first representation or to the second representation,
outputting and/or storing the synthetic contrast-enhanced representation of the examination region of the examination object and/or transmitting the synthetic contrast-enhanced representation to a separate computer system.

25. Use according to claim 24, wherein the radiological examination method is a magnetic resonance imaging examination or a computed tomography examination and wherein the contrast agent comprises
a Gd³⁺ complex of a compound of the formula (I) 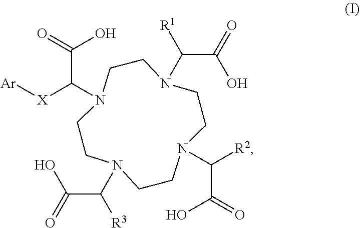
where
Ar is a group selected from 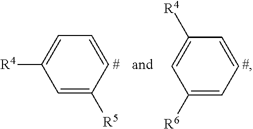
where #is the linkage to X,
X is a group selected from
CH₂, (CH₂)₂, (CH₂)₃, (CH₂)₄ and *—(CH₂)₂—O—CH₂—^{#},
where * is the linkage to Ar and # is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof, or
a Gd³⁺ complex of a compound of the formula (II) 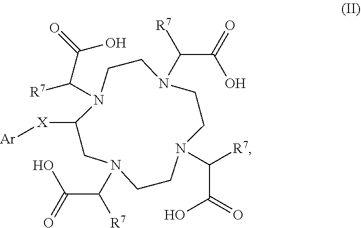
where
Ar is a group selected from 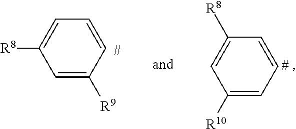
where # is the linkage to X,
X is a group selected from CH₂, (CH₂)₂, (CH₂)₃, (CH₂)₄ and *—(CH₂)₂—O—CH₂—#, where * is the linkage to Ar and # is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof, or
the contrast agent comprises one of the following substances:
gadolinium(III) 2-[4,7, 10-tris(carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis [1-carboxylato-4-(2,3-dihydroxypropylamino)-4-oxobutyl]-3,6,9,15-tetrazabicyclo[9.3.1] pentadeca-1 (15), 11, 13-trien-6-yl]-5-(2,3-dihydroxypropylamino)-5-oxopentanoate,
dihydrogen [(±)-4-carboxy-5,8, 11-tris(carboxymethyl)-1-phenyl-2-oxa-5,8,11-triazatridecan-13-oato(5-)]gadolinate (2-),
tetragadolinium [4, 10-bis(carboxylatomethyl)-7-{3,6, 12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino)acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate,
gadolinium 2,2′,2″-{10-[(1R)-1-carboxy-2-{4-[2-(2-ethoxyethoxy)ethoxy]phenyl}ethyl]-1,4,7, 10-tetraazacyclododecane-1,4,7-triyl}triacetate,
gadolinium (2S,2′S,2″S)-2,2′,2″-{10- [(1S)-1-carboxy-4-{4-[2-(2-ethoxyethoxy)ethoxy]phenyl}butyl]-1,4,7,10-tetraazacyclododecane-1,4,7-triyl}tris (3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis (carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis (2-oxido-2-oxoethyl)-1,4,7,10-tetrazacyclododec-1-yl]acetate,
gadolinium(III) 2,2′,2″-(10- ((2R,3S)-1,3,4-trihydroxybutan-2-yl)-1,4,7,10-tetraazacyclododecane-1,4,7-triyl)triacetate,
gadolinium 2,2′,2″-{(2S)-10-(carboxymethyl)-2-[4-(2-ethoxyethoxy)benzyl]-1,4,7,10-tetraazacyclododecane-1,4,7-triyl}triacetate,
gadolinium 2,2′,2″-[10-(carboxymethyl)-2-(4-ethoxybenzyl)-1,4,7,10-tetraazacyclododecane-1,4,7-triyl]triacetate.

26. Kit comprising a computer program product according to claim 23 and a contrast agent, wherein the contrast agent preferably comprises
a Gd³⁺ complex of a compound of the formula (I) 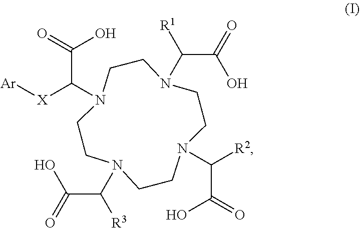
where
Ar is a group selected from 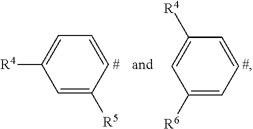
where # is the linkage to X,
X is a group selected from
CH₂, (CH₂)₂, (CH₂)₃, (CH₂)₄ and *—(CH₂)₂—O—CH₂—^{#},
where * is the linkage to Ar and # is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof, or
a Gd³⁺ complex of a compound of the formula (II) 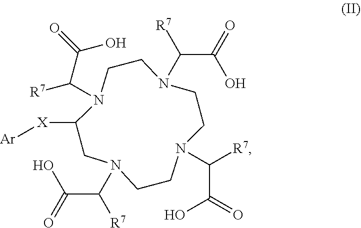
where
Ar is a group selected from 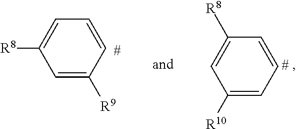
where # is the linkage to X,
X is a group selected from CH₂, (CH₂)₂, (CH₂)₃, (CH₂)₄ and *—(CH₂)₂—O—CH₂—#, where * is the linkage to Ar and # is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from
C₂—C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof, or
the contrast agent comprises one of the following substances:
gadolinium(III) 2-[4,7,10-tris (carboxymethyl)-1,4,7,10-tetrazacyclododec-1-yl]acetic acid,
gadolinium(III) ethoxybenzyldiethylenetriaminepentaacetic acid,
gadolinium(III) 2-[3,9-bis [1-carboxylato-4-(2,3-dihydroxypropylamino)-4-oxobutyl]-3,6,9,15-tetrazabicyclo[9.3.1] pentadeca-1 (15), 11,13-trien-6-yl]-5-(2,3-dihydroxypropylamino)-5-oxopentanoate,
dihydrogen [(±)-4-carboxy-5,8, 11-tris(carboxymethyl)-1-phenyl-2-oxa-5,8,11-triazatridecan-13-oato (5-)]gadolinate (2-),
tetragadolinium [4, 10-bis (carboxylatomethyl)-7-{3,6,12,15-tetraoxo-16-[4,7,10-tris(carboxylatomethyl)-1,4,7,10-tetraazacyclododecan-1-yl]-9,9-bis({[({2-[4,7,10-tris(carboxylatomethyl)-1,4,7,10-tetraazacyclododecan-1-yl]propanoyl}amino) acetyl]-amino}methyl)-4,7,11,14-tetraazahepta-decan-2-yl}-1,4,7,10-tetraazacyclododecan-1-yl]acetate,
2,2′,2″-(10-{1-carboxy-2-[2-(4-ethoxyphenyl)ethoxy]ethyl}-1,4,7,10-tetraazacyclododecane-1,4,7-triyl)triacetate,
gadolinium 2,2′,2″-{10-[1-carboxy-2-{4-[2-(2-ethoxyethoxy) ethoxy]phenyl}ethyl]-1,4,7,10--tetraazacyclododecane-1,4,7-triyl}triacetate,
gadolinium 2,2′,2″-{10-[(1R)-1-carboxy-2-{4-[2-(2-ethoxyethoxy) ethoxy]phenyl}ethyl]-1,4,7,10-tetraazacyclododecane-1,4,7-triyl}triacetate,
gadolinium (2S,2′S,2″S)-2,2′,2″-{10- [(1S)-1-carboxy-4-{4-[2-(2-ethoxyethoxy) ethoxy]phenyl}butyl]-1,4,7,10-tetraazacyclododecane-1,4,7-triyl}tris (3-hydroxypropanoate)
gadolinium 2,2′,2″-{10-[(1S)-4-(4-butoxyphenyl)-1-carboxybutyl]-1,4,7,10-tetraazacyclododecane-1,4,7-triyl}triacetate,
gadolinium(III) 5,8-bis(carboxylatomethyl)-2-[2-(methylamino)-2-oxoethyl]-10-oxo-2,5,8,11-tetraazadodecane-1-carboxylate hydrate
gadolinium(III) 2-[4-(2-hydroxypropyl)-7,10-bis (2-oxido-2-oxoethyl)-1,4,7,10-tetrazacyclododec-1-yl]acetate,
gadolinium(III) 2,2′,2″-(10-(2R,3S)-1,3,4-trihydroxybutan-2-yl)-1,4,7,10-tetraazacyclododecane-1,4,7-triyl)triacetate,
gadolinium 2,2′,2″-{(2S)-10-(carboxymethyl)-2-[4-(2-ethoxyethoxy) benzyl]-1,4,7,10-tetraazacyclododecane-1,4,7-triyl}triacetate,
gadolinium 2,2′,2″-[10-(carboxymethyl)-2-(4-ethoxybenzyl)-1,4,7,10-tetraazacyclododecane-1,4,7-triyl]triacetate.
